(19) United States
(12) Patent Application Publication
(10) Pub. No.: US 2016/0363571 A1
(43) Pub. Date: Dec. 15, 2016

(54) SIMULATION SYSTEM FOR SIMULATING PARTICULATE MATTER EMISSIONS OF AN ENGINE

(71) Applicant: Caterpillar Inc., Peoria, IL (US)

(72) Inventor: Radhakrishnan Purushothaman, Peoria, IL (US)

(73) Assignee: Caterpillar Inc., Peoria, IL (US)

(21) Appl. No.: 15/244,639

(22) Filed: Aug. 23, 2016

(51) Int. Cl. G01N 33/00; G01M 15/10; G01N 15/06; F01N 3/20; F01N 13/08

(52) U.S. Cl. CPC G01N 33/0006; F01N 3/20; F01N 13/082; G01N 15/06; G01M 15/102; F01N 2560/05; G01N 2015/0046

(57) ABSTRACT

A simulation system for simulating particulate matter emissions of an engine is provided. The simulation system comprises a soot generator and an exhaust assembly. The soot generator generates soot. The exhaust assembly is positioned downstream to the soot generator in an exhaust flow direction. The exhaust assembly includes a primary diluter, a catalytic stripper, at least one secondary diluter, a residence chamber, and a tail pipe, which are positioned sequentially in the exhaust flow direction. Components of the exhaust assembly are in fluid communication with the other components positioned upstream or downstream to them. The tail pipe includes a first sample port and a second sample port. The first sample port is fluidly connected to a reference measuring instrument and the second sample port being connected to a particulate measuring instrument.

### TECHNICAL FIELD

The present disclosure generally relates to a simulation system for simulating particulate matter emissions of an engine. More particularly, the present disclosure relates to use of the simulation system, for checking accuracy and calibration of one or more particulate measuring instruments.

### BACKGROUND

Exhaust gas flowing out of an engine, such as a diesel engine, is a complex mixture of gaseous components and particulate matter emissions. Among other unwanted components, the particulate matter emissions include carbon as a main component, also known as soot. A high amount of the soot in exhaust gas is harmful for human beings and environment. Therefore, standards are defined to limit the amount and size of the soot in the particulate matter emissions. Also, the particulate matter emissions flowing out of the engine are required to be tested for meeting various regulatory requirements and for optimizing engine performance. A variety of particulate measuring instruments are used for determining the particulate matter emissions in the exhaust gas. These particulate measuring instruments are required to be periodically calibrated and checked for accuracy. Such calibration and checking procedures are generally performed in a laboratory environment.

Typically, a diesel engine is employed in the laboratory environment for generating particulate matter emissions, in order to calibrate and check accuracy of the particulate measuring instruments. However, using the diesel engine in the laboratory environment may cause inconvenience to users. For example, the diesel engine generates a lot of noise and requires dampening, which may cause inconvenience to the users. Furthermore, a weight of the diesel engine makes the entire system bulky and cumbersome, requiring rig construction for proper mounting of the diesel engine. Additionally, usage of the diesel engine requires an aftertreatment arrangement for treating the volatile components in the particulate matter emissions, in order to check accuracy and calibrate the particulate measuring instruments. Therefore, employing the diesel engine increases operating and maintenance costs in the laboratory environment.

Hence, there is a need for a system to simulate the particulate matter emissions, in order to check accuracy and calibrate the particulate measuring instruments in the laboratory environment.

### SUMMARY OF THE DISCLOSURE

In an aspect of the present disclosure, a simulation system for simulating particulate matter emissions of an engine is provided. The simulation system comprises a soot generator and an exhaust assembly. The soot generator generates soot. The exhaust assembly is positioned downstream to the soot generator in an exhaust flow direction. The exhaust assembly includes a primary diluter, a catalytic stripper, at least one secondary diluter, a residence chamber, and a tail pipe. The primary diluter is in fluid communication with the soot generator. The catalytic stripper is fluidly connected to the first diluter. The catalytic stripper is positioned downstream to the first diluter in the exhaust flow direction. The secondary diluter is fluidly connected to the catalytic stripper. The secondary diluter is positioned downstream to the catalytic stripper in the exhaust flow direction. The residence chamber is fluidly connected to the secondary diluter. The residence chamber is positioned downstream to the secondary diluter in the exhaust flow direction. The tail pipe is fluidly connected to and positioned downstream to the residence chamber in the exhaust flow direction. The tail pipe includes a first sample port and a second sample port. The first sample port is fluidly connected to a reference measuring instrument, and the second sample port being connected to a particulate measuring instrument.

### BRIEF DESCRIPTION OF THE DRAWINGS

FIG. 1 is a perspective view of a simulation system, in accordance with the concepts of the present disclosure:

FIG. 2 is a block diagram of the simulation system of FIG. 1, illustrating a number of particulate measuring instruments under calibration and accuracy check, in accordance with the concepts of the present disclosure; and

FIG. 3 is a flow chart of a method of simulating particulate matter emissions, in accordance with the concepts of the present disclosure.

### DETAILED DESCRIPTION

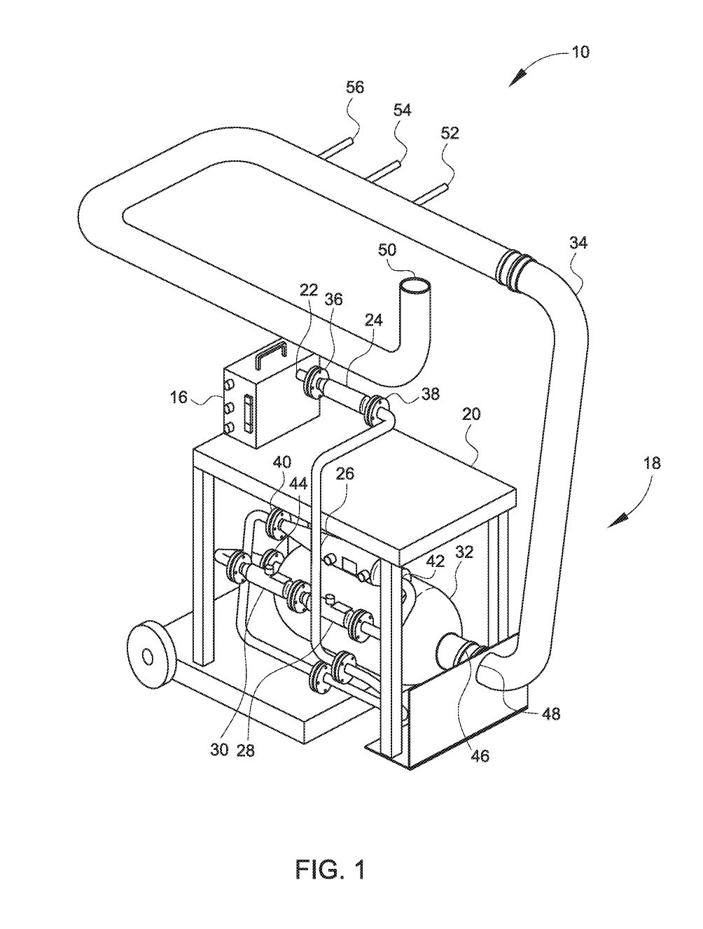

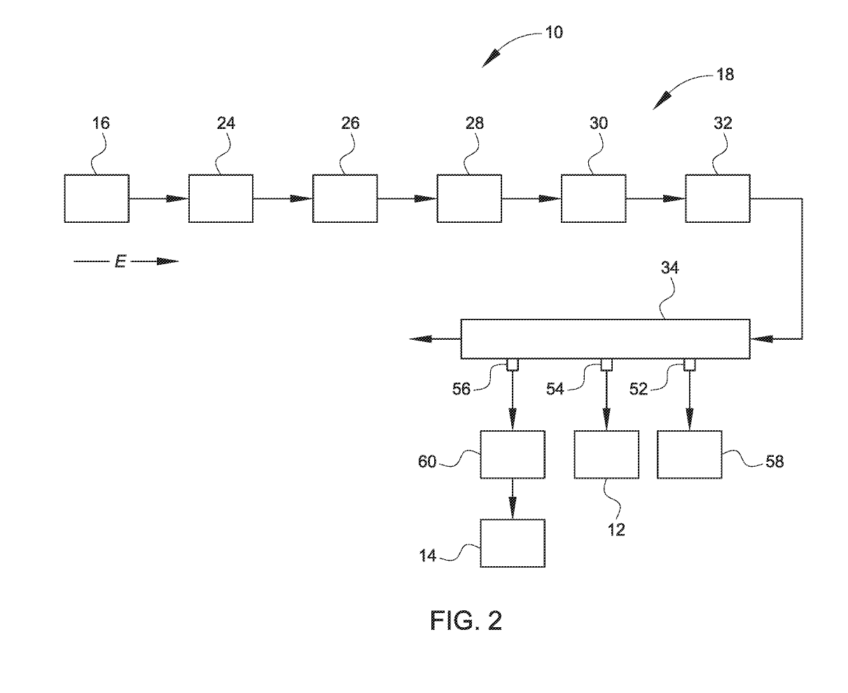

Referring to FIGS. 1 and 2, a simulation system 10 for simulating particulate matter emissions of an engine (not shown) is shown. More specifically, the simulation system 10 simulates particulate matter emissions, which is then used for checking accuracy and calibration of one or more particulate measuring instruments 12, 14 (shown in FIG. 2). The simulation system 10 includes a soot generator 16 and an exhaust assembly 18, suitably installed on a support frame 20.

The soot generator 16 of the simulation system 10 generates soot. The soot is a mass of impure carbon particles formed within a diffusion flame, which is generated by pyrolysis of a hydrocarbon fuel. More specifically, the soot generator 16 burns the hydrocarbon fuel and then supply limited amount of air at a flame front, in order to generate the soot. In an embodiment, the soot generated is stabilized by mixing with a quenching gas. The soot generator 16 further employs a flow controller (not shown), which alters a size and amount of the soot by varying a concentration of one or more of the hydrocarbon fuel, the air, and the quenching gas. Therefore, the soot generator 16 is capable of generating same amount of the soot as that of the engine (not shown). The soot generator 16 may be contemplated to generate same amount of the soot as that of various types of the engine (not shown). Examples of the engine may include, but is not limited to, a diesel engine, a gaseous engine, and a gasoline engine. Further, the soot generator 16 includes a generator outlet 22, which ejects and supplies the soot to the exhaust assembly 18.

The exhaust assembly 18 is positioned downstream to the soot generator 16, in an exhaust flow direction E. The exhaust assembly 18 is adapted to treat the soot, in order to simulate the particulate matter emissions of the engine (not shown). The exhaust assembly 18 includes a primary diluter 24, a catalytic stripper 26, two secondary diluters 28, 30, a residence chamber 32, and a tail pipe 34. The primary diluter 24, the catalytic stripper 26, the secondary diluters 28, 30, the residence chamber 32, and the tail pipe 34 are in sequential fluid communication with each other, to facilitate multiple level treatment of the soot. Usage of a number of conduits to facilitate a fluid communication between various components of the exhaust assembly 18 may be contemplated.

The primary diluter 24 is fluidly connected to the soot generator 16 and is disposed downstream to the soot generator 16 in the exhaust flow direction E. The primary diluter 24 includes a diluter inlet 36 and a diluter outlet 38. The diluter inlet 36 of the primary diluter 24 is fluidly connected to the generator outlet 22 of the soot generator 16. The primary diluter 24 is adapted to lower a concentration of the inflowing soot by a first value. In an embodiment, the primary diluter 24 is fluidly connected to an airflow supply (not shown), to receive a supply of filtered air. The primary diluter 24 mixes the filtered air to the inflowing soot, in order to lower the concentration of the inflowing soot by the first value.

The catalytic stripper 26 is fluidly connected to the soot generator 16 and is positioned downstream to the primary diluter 24 in the exhaust flow direction E. The catalytic stripper 26 includes a stripper inlet 40 and a stripper outlet 42. The stripper inlet 40 of the catalytic stripper 26 is fluidly connected to the diluter outlet 38 of the primary diluter 24. In an embodiment, the catalytic stripper 26 employs a catalyst layer (not shown) to oxidize the volatile compounds in the inflowing soot. Examples of the catalyst layer (not shown) include, but are not limited to, platinum, rhodium, or palladium. More specifically, the catalytic stripper 26 heats up the inflowing soot above 350 degree Celsius and then pass the soot through the catalyst layer (not shown), to oxidize the volatile compounds in the inflowing soot.

The secondary diluters 28, 30 are fluidly connected to and positioned downstream to the catalytic stripper 26 in the exhaust flow direction E. In the current embodiment, the exhaust assembly 18 employs two secondary diluters 28, 30, namely a first diluter 28 and a second diluter 30. The first diluter 28 and the second diluter 30 are sequentially fluidly connected with each other, in the exhaust flow direction E. The first diluter 28 is fluidly connected to the stripper outlet 42 of the catalytic stripper 26. The first diluter 28 and the second diluter 30, in conjunction, are adapted to lower the concentration of the inflowing soot by a second value. Although, the present disclosure contemplates usage of two diluters as the secondary diluters 28, 30, usage of a singular diluter, to lower the concentration of the inflowing soot by the second value, may also be contemplated. Usage of multiple diluters as the secondary diluters 28, 30, to lower the concentration of the inflowing soot by the second value, may also be contemplated.

The residence chamber 32 is fluidly connected to and positioned downstream to the second diluter 30 in the exhaust flow direction E. The residence chamber 32 includes a residence chamber inlet 44 and a residence chamber outlet 46. The residence chamber inlet 44 is fluidly connected to the second diluter 30. The residence chamber 32 is adapted to stabilize the concentration of inflowing soot, to prevent agglomeration of the soot. More specifically, the residence chamber 32 reduces a speed of the soot, in order to stabilize the inflowing soot.

The tail pipe 34 is fluidly connected to and is positioned downstream to the residence chamber 32 in the exhaust flow direction E. The tail pipe 34 includes a first end 48, a second end 50, and three sample ports 52, 54, 56. The first end 48 of the tail pipe 34 is fluidly connected to the residence chamber 32. The second end 50 of the tail pipe 34 vents the soot from the simulation system 10. In a preferred embodiment, the tail pipe 34 includes three sample ports 52, 54, 56, namely a first sample port 52, a second sample port 54, and a third sample port 56. Although, the present disclosure describes three sample ports 52, 54, 56 on the tail pipe 34, multiple sample ports on the tail pipe 34, may also be contemplated.

Furthermore, the exhaust assembly 18 of the simulation system 10 sequentially passes the soot, as generated by the soot generator 16, through each of the primary diluter 24, the catalytic stripper 26, the secondary diluters 28, 30, and the residence chamber 32, to generate the particulate matter emissions in the tail pipe 34. The particulate matter emissions, as generated by the simulation system 10, are further used for calibration and checking accuracy of the particulate measuring instruments 12, 14 (shown in FIG. 2).

FIG. 2 illustrates a block diagram of the simulation system 10, illustrating the particulate measuring instruments 12, 14. In the current embodiment, the simulation system 10 is described to be used for calibration and checking accuracy of two particulate measuring instruments 12, 14, namely a first particulate measuring instrument 12 and a second particulate measuring instrument 14. The first particulate measuring instrument 12 is a charge ineffective measuring instrument, while the second particulate measuring instrument 14 is a charge effective measuring instrument. Examples of the first particulate measuring instrument 12 and the second particulate measuring instrument 14 may include, but not limited to, TSI Scanning Mobility Particle Sizer Spectrometer (SMPS), Engine Exhaust Condensation Particle Counter (EECPC), HORIBA Solid Particle Counting System (SPCS), TSI Engine Exhaust Particle Sizer (EEPS) spectrometer, Cambustion DMS 500, DEKATI Electrical Low Pressure Impactor (ELPI), AVL Microsoot, and AVL smoke meters.

Furthermore, a reference measuring instrument 58 is fluidly connected to the first sample port 52 of the tail pipe 34. The first particulate measuring instrument 12 and the second particulate measuring instrument 14 are fluidly connected to the second sample port 54 and the third sample port 56 of the tail pipe 34, respectively. In an embodiment, the third sample port 56 is fluidly connected to the second particulate measuring instrument 14, via a charge neutralizer 60. The charge neutralizer 60 neutralizes a charge in the soot, for precise calibration and checking accuracy of the second particulate measuring instrument 14. Each of the reference measuring instrument 58, the first particulate measuring instrument 12, and the second particulate measuring instrument 14 receives a portion of particulate matter emissions from the first sample port 52, the second sample port 54, and the third sample port 56, respectively. Further, each of the reference measuring instrument 58, the first particulate measuring instrument 12, and the second particulate measuring instrument 14 provides an indication of measurement of the soot. A measurement value of the reference measuring instrument 58 is compared with a measurement value of the first particulate measuring instrument 12 and the second particulate measuring instrument 14, in order to check accuracy and calibrate the first particulate measuring instrument 12 and the second particulate measuring instrument 14, respectively.

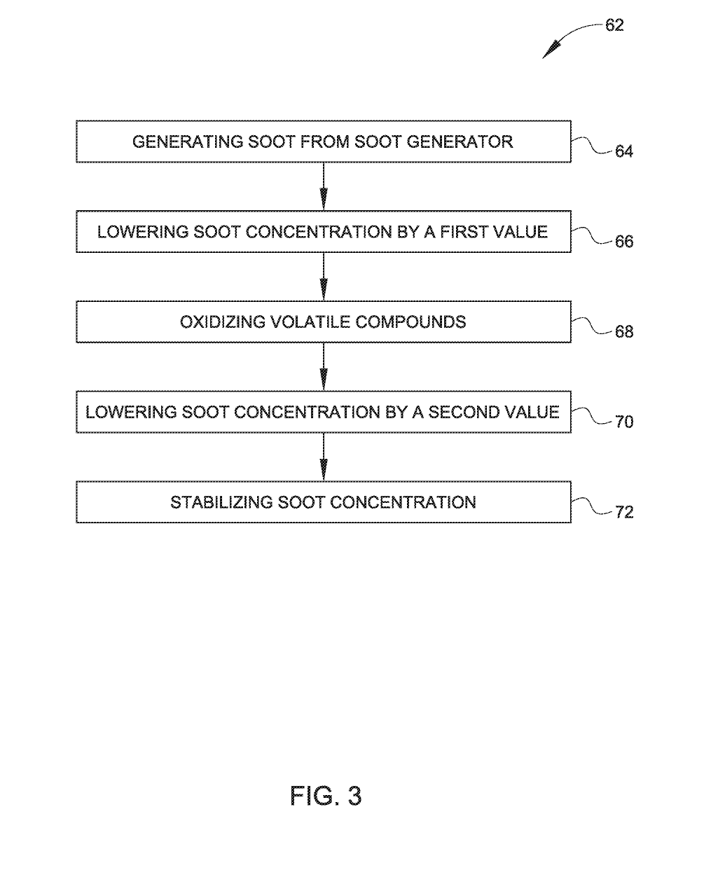

FIG. 3 illustrates a flowchart of a method 62 of simulation of the particulate matter emissions. The method 62 is employed to sample the portion of the particulate matter emissions, to check accuracy and calibrate the particulate measuring instruments 12, 14. The method 62 initiates at step 64.

At step 64, the soot generator 16 generates the soot. More specifically, the soot generator 16 performs pyrolysis of the hydrocarbon fuel to generate the soot. The soot is then passed through the primary diluter 24 of the exhaust assembly 18. The method 62 then proceeds to step 66.

At step 66, the primary diluter 24 lowers the concentration of the soot by the first value. More specifically, the primary diluter 24 receives the soot from the soot generator 16 and mixes the filtered air with the inflowing soot, to lower the concentration of the soot by the first value. The soot is then passed through the catalytic stripper 26. The method 62 then proceeds to step 68.

At step 68, the catalytic stripper 26 oxidizes the volatile compounds in the inflowing soot. More specifically, the soot is allowed to pass through the catalyst layer (not shown) of the catalytic stripper 26, which oxidizes the volatile compounds in the inflowing soot. The soot is then passed through the secondary diluters 28, 30. The method 62 proceeds to step 70.

At step 70, the secondary diluters 28, 30 lower the concentration of the inflowing soot by the second value. More specifically, the secondary diluters 28, 30 mix an additional amount of filtered air to the inflowing soot. The secondary diluters 28, 30 lower the concentration of the soot, such that the soot is transformed to the particulate matter emission. The particulate matter emissions so produced is then passed through the residence chamber 32. The method 62 then proceeds to step 72.

At step 72, the residence chamber 32 stabilizes the soot. More specifically, the residence chamber 32 decelerates the particulate matter emissions, therefore, reduces agglomeration of the soot in the particulate matter emissions. Therefore, particulate matter emissions are generated in the tail pipe 34, which may be used for calibration and checking accuracy of the particulate measuring instruments 12, 14.

### INDUSTRIAL APPLICABILITY

In operation, the simulation system 10 disclosed herein simulates the particulate matter emissions. Initially, the soot generator 16 generates the same amount of soot as that of the engine (not shown). The soot thus produced is passed through each of the primary diluter 24, the catalytic stripper 26, the secondary diluters 28, 30, and the residence chamber 32 of the exhaust assembly 18, to simulate the particulate matter emissions of the engine (not shown).

Initially, the soot is passed through the primary diluter 24. The primary diluter 24 lowers the concentration of the soot by the first value. The soot is then passed through the catalytic stripper 26. The catalytic stripper 26 reduces an amount of the volatile compounds in the soot. The soot is then passed through the secondary diluters 28, 30. The secondary diluters 28, 30 lower the concentration of the soot by the second value. The soot is then passed through the residence chamber 32. The residence chamber 32 stabilizes the soot to form the particulate matter emissions, for calibration and checking accuracy of the particulate measuring instrument 12, 14. Thereafter at least a portion of the particulate matter emissions is passed through each of the reference measuring instrument 58, the first particulate measuring instrument 12, and the second particulate measuring instrument 14, for calibration and checking accuracy the first particulate measuring instrument 12 and the second particulate measuring instrument 14. For calibration and checking accuracy, the measurement value of the reference measuring instrument 58 is compared with the measurement value of the first particulate measuring instrument 12 and the second particulate measuring instrument 14.

In the current disclosure, as the simulation system 10 employs the soot generator 16 and the exhaust assembly 18 to simulate the particulate matter emissions, a need of conventional engines (not shown) for generating the particulate matter emissions is eliminated. This eliminates a need of additional support structures and associated after-treatment systems in the laboratory environment.

As the disclosed simulation system 10 employs relatively lesser structures, overall cost of simulation system 10 for checking accuracy and calibration of the particulate measuring instruments 12, 14 is reduced. Also, the disclosed simulation system 10 is relatively less noisy, less bulky, and easy to use. Moreover, as the catalytic stripper 26 oxidizes the volatile compounds in the soot, the particulate measuring instruments 12, 14 are precisely checked for measurement of solid particles in the soot. Therefore, the disclosed simulation system 10 conforms to latest European regulations.

While aspects of the present disclosure have been particularly shown and described with reference to the embodiments above, it will be understood by those skilled in the art that various additional embodiments may be contemplated by the modification of the disclosed machines, systems, and methods without departing from the spirit and scope of the disclosure. Such embodiments should be understood to fall within the scope of the present disclosure as determined based upon the claims and any equivalents thereof.

What is claimed is:

1. A simulation system for simulating particulate matter emissions of an engine, the simulation system comprising:
a soot generator adapted to generate soot;
an exhaust assembly positioned downstream to the soot generator in an exhaust flow direction, the exhaust assembly including:
a primary diluter in fluid communication with the soot generator;
a catalytic stripper fluidly connected to the first diluter, the catalytic stripper being positioned downstream to the first diluter in the exhaust flow direction;
at least one secondary diluter fluidly connected to the catalytic stripper, the at least one secondary diluter being positioned downstream to the catalytic stripper in the exhaust flow direction;
a residence chamber fluidly connected to the at least one secondary diluter, the residence chamber being positioned downstream to the at least one secondary diluter in the exhaust flow direction; and
a tail pipe fluidly connected to and positioned downstream to the residence chamber in the exhaust flow direction.

2. The simulation system of claim 1, wherein the tail pipe includes a first sample port and a second sample port, the first sample port being fluidly connected to a reference measuring instrument, the second sample port being connected to a particulate measuring instrument.
